SEQUENCE LISTING (28 sequences)

SEQ ID NO: 1 - protein, 200 aa, Homo sapiens
MAFTEHSPLTPHRRDLCSRSIWLARKIRSDLTALTESYVKHQGLNKNINLDSADGMPVASTDQWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWTVRSIHDLRFISSHQTGIPARGSHYIANNKKM

SEQ ID NO: 2 - protein, 200 aa, Artificial Sequence
  Synthetic
MAFTEHSPLTPHRRDLCSRSIWLARKIRSDLTALTESYVKHQGLNKNINLDSVDGVPVASTDRWSEMTEAERLQENLQAYRTFQGMLTKLLEDQRVHFTPTEGDFHQAIHTLLLQVSAFAYQLEELMVLLEQKIPENEADGMPATVGDGGLFEKKLWGLKVLQELSQWTVRSIHDLRVISSHQMGISALESHYGAKDKQM

SEQ ID NO: 3 - protein, 185 aa, Artificial Sequence
  synthetic
MAFTEHSPLTPHRRDLASRSIWLARKIRSDLTALTESYVKHQGLNKNINLDSADGMPVASTDRWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWTVRSIHDLRFISSHQTG

SEQ ID NO: 4 - protein, 184 aa, Artificial Sequence
  Synthetic
AFTEHSPLTPHRRDLASRSIWLARKIRSDLTALTESYVKHQGLNKNINLDSADGMPVASTDRWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWTVRSIHDLRFISSHQTG

SEQ ID NO: 5 - protein, 187 aa, Homo sapiens
MAFTEHSPLTPHRRDLASRSIWLARKIRSDLTALTESYVKHQGLNKNINLDSADGMPVASTDRWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWTVRSIHDLRFISSHQTGIP

SEQ ID NO: 6 - protein, 187 aa, Homo sapiens
MAFTEHSPLTPHRRDLCSRSIWLARKIRSDLTALTESYVKHQGLNKNINLDSADGMPVASTDQWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWTVRSIHDLRFISSHQTGIP

SEQ ID NO: 7 - protein, 187 aa, Homo sapiens
MAFTEHSPLTPHRRDLCSRSIWLARKIRSDLTALTESYVKHQGLNKNINLDSADGMPVASTDRWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWTVRSIHDLRFISSHQTGIP

SEQ ID NO: 8 - protein, 187 aa, Homo sapiens
MAFTEHSPLTPHRRDLASRSIWLARKIRSDLTALTESYVKHQGLNKNINLDSADGMPVASTDQWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWTVRSIHDLRFISSHQTGIP

SEQ ID NO: 9 - protein, 13 aa, Homo sapiens
GDFHQAIHTLLLQ

SEQ ID NO: 10 - protein, 13 aa, Homo sapiens
ARKIRSDLTALTE

SEQ ID NO: 11 - protein, 13 aa, Homo sapiens
NKNINLDSADGMP

SEQ ID NO: 12 - protein, 18 aa, Homo sapiens
AERLQENLQAYRTFHVLL

SEQ ID NO: 13 - protein, 15 aa, Homo sapiens
YRTFHVLLARLLEDQ

SEQ ID NO: 14 - protein, 15 aa, Homo sapiens
LARLLEDQQVHFTPT

SEQ ID NO: 15 - protein, 12 aa, Homo sapiens
TEGDFHQAIHTL

SEQ ID NO: 16 - protein, 15 aa, Homo sapiens
IHTLLLQVAAFAYQI

SEQ ID NO: 17 - protein, 13 aa, Homo sapiens
QIEELMILLEYKI

SEQ ID NO: 18 - protein, 13 aa, Homo sapiens
EYKIPRNEADGMP

SEQ ID NO: 19 - protein, 13 aa, Homo sapiens
VGDGGLFEKKLWG

SEQ ID NO: 20 - protein, 13 aa, Homo sapiens
LWGLKVLQELSQW

SEQ ID NO: 21 - protein, 13 aa, Homo sapiens
SQWTVRSIHDLRF

SEQ ID NO: 22 - protein, 13 aa, Homo sapiens
QELSQWTVRSIHD

SEQ ID NO: 23 - protein, 10 aa, Homo sapiens
LRFISSHQTG

SEQ ID NO: 24 - protein, 13 aa, Homo sapiens
QELSQWTVRSIHD

SEQ ID NO: 25 - protein, 22 aa, Homo sapiens
EETCLVKIITGLLEFEVYLEYL

SEQ ID NO: 26 - protein, 14 aa, Homo sapiens
RAVQMSTKVLIQFL

SEQ ID NO: 27 - protein, 20 aa, Homo sapiens
AGCLSQLHSGLFLYQGLLQA

SEQ ID NO: 28 - protein, 24 aa, Homo sapiens
PTLDTLQLDVADFATTIWQQMEEL

(19) United States
(12) Patent Application Publication
(10) Pub. No.: US 2005/0069987 A1
(43) Pub. Date: Mar. 31, 2005

(54) Modified ciliary neurotrophic factor polypeptides with reduced antigenicity

(76) Inventors: Thomas J. Daly, New City, NY (US); Margaret Karow, Putnam Valley, NY (US)

Correspondence Address: REGENERON PHARMACEUTICALS, INC, 777 OLD SAW MILL RIVER ROAD, TARRYTOWN, NY, 10591, US

(21) Appl. No.: 10/954,466

(22) Filed: Sep. 30, 2004

Related U.S. Application Data
(60) Provisional application No. 60/507,157, filed on Sep. 30, 2003.
(60) Provisional application No. 60/567,060, filed on Apr. 30, 2004.

(51) Int. Cl. C12P 21/02; C07H 21/04; C07K 14/525

(52) U.S. Cl. 435/69.5; 530/351; 536/23.5; 435/320.1; 435/325

(57) ABSTRACT

Modified human ciliary neurotrophic factor (hCNTF) molecules having reduced antigeniticy relative to non-modified hCNTF molecules are describes, as well as methods for production and methods of use, especially in the treatment of obesity and obesity-related diseases or conditions.

### CROSS-REFERENCE TO RELATED APPLICATIONS

This application claims the benefit under 35 USC § 119(e) of U.S. provisional applications 60/507,157 filed 30 Sep. 2003 and 60/567,060 filed 30 Apr. 2004, which applications are herein specifically incorporated by reference in their entirety.

### BACKGROUND OF THE INVENTION

1. Field of the Invention

The invention encompasses modified ciliary neurotrophic factor (CNTF) molecules with reduced antigenicity.

2. Description of Related Art

U.S. Pat. Nos. 6,472,178 and 6,565,869 describe modified ciliary neurotrophic factor (CNTF) molecules useful for treatment of a number of diseases, including neurological diseases, obesity, and diabetes. Human CNTF is shown in SEQ ID NO:1. Variants of human CNTF are shown as follows: hCNTF (Q63R) SEQ ID NO:2; Axokine™(“Ax-15”; hCNTF C17A Q63RΔ15) (SEQ ID NO:3 or SEQ ID NO:4); Ax-13 (hCNTF C17A Q63R Δ13) (SEQ ID NO:5); hCNTFΔ13 (SEQ ID NO:6); hCNTF Q63RΔ13 (SEQ ID NO:7); hCNTF C17AΔ13 (SEQ ID NO:8).

Methods for protein mutagenesis are known to the art, see for example, Cunningham et al. (1989) Science 243:1330-1336 and 244:1081-1085; Masiakowski et al. (1991) J. Neurochem. 57:1003-1012.). Panyotatos et al. (1993) J. Biol. Chem. 268:19000-19003 and (1994) Biochemistry 33:5813-5818, describe a mutation at position 63 of human CNTF greatly enhanced the affinity of human CNTF for soluble CNTF receptor alpha (sCNTFRα) as well as increasing its biological potency.

Human clinical trials using recombinant human CNTF (rHCNTF) have shown that a majority of patients developed neutralizing antibodies (ALS CNTF Treatment Study Group (1996) Neurology 46:1244-1249).

### SUMMARY OF THE INVENTION

The present invention is based, in part, on identification of a peptide within a human ciliary neurotrophic factor (hCNTF) which is highly antigenic. Identification of this peptide allows generation of modified hCNTF molecules with reduced antigenicity when administered therapeutically to a human subject. The present invention is further based in part on the generation of hCNTF molecules with improved properties such as improved PK and stability.

Accordingly, in a first aspect, the invention features a human CNTF (hCNTF) molecule having reduced antigenicity and/or improved properties relative to a non-modified hCNTF molecule. In a first embodiment, the modified hCNTF molecule having reduced antigenicity comprises a molecule modified by one or more of the modifications of group I comprising at least one amino acid modification within one or more of a peptide selected from the group consisting of 103-115 (SEQ ID NO:9),24-36 (SEQ ID NO:10), 45-57 (SEQ ID NO:11), 70-87 (SEQ ID NO:12),80-NO:13), 87-101 (SEQ ID NO:14), 101-112 (SEQ ID NO:15), 109-123 (SEQ ID NO:16) 122-134 (SEQ ID NO:17), 131-143 (SEQ ID NO:18), 146-158 (SEQ ID NO:19), 156-168 (SEQ ID NO:20), 166-178 (SEQ ID NO:21), 163-175 (SEQ ID NO:22), 176-185 (SEQ ID NO:23), all of SEQ ID NO:1, or the equivalent peptide of a hCNTF variant. Non-limiting examples of hCNTF variants are shown in SEQ ID NOs:2-8. The invention may be practiced with other hCNTF variants,-for example, hCNTFΔ15; hCNTFQ63RΔ15; hCNTF C17AΔ15, etc. When the parent hCNTF molecule is SEQ ID NO:4 (Axokine™ without N-terminal Met), the corresponding amino acid positions are reduced by one, e.g., the peptide of SEQ ID NO:9 is found at amino acids 105-116.

When the modified hCNTF comprises at least one modification within the peptide of SEQ ID NO:9 (amino acids 103-115), or the equivalent peptide of an hCNTF variant, at least one modification is at Phe at position 105 (Phe105), His110, Leu112 and/or Leu113. In one preferred embodiment, the PhelO5 is replaced with any one of Ala, Lys, Asn, Gln, Ser, Thr, Glu, Pro, Arg, Asp, Gly, His, or Cys. Most preferably, the Phe105 is replaced with Ala (Phe105→Ala). In another preferred embodiment, the His110 is replaced with Lys, Glu, Ala, Ile, Leu, Trp, Tyr, Asp, Gln, Ser, Thr, or Cys. In a most preferred embodiment, His110→Lys, Glu, or Asp. In yet another preferred embodiment, the Leu112→Glu. In yet another preferred embodiment, the Leu113→Ala, Ile, Tyr, Asn, or Ser. In other specific embodiments, the modified hCNTF molecule having reduced antigenicity comprises a modification of Phe105+His110; Phe105+His110+Leu112; Phe105+His110+Leu113; or Phe105+His110+Leu112+Leu113.

When the modified hCNTF comprises at least one modification within peptide of SEQ ID NO:12, or the equivalent peptide of an hCNTF variant, at position Leu77, Tyr80, Phe83, a preferred modification is Leu77→Ala, Tyr80→Ala, and Phe83→Ala.

When the modified hCNTF comprises at least one modification within the peptide of SEQ ID NO:13 (amino acids 80-94), or the equivalent peptide of an hCNTF variant, at least one modification is at Tyr80, Phe83, Ala88 and/or Leu90. While these amino acids may be substituted with any amino acid, in one preferred embodiment, Tyr80→Ala, Phe83→Ala, Ala88→Asn, and Leu→90Ala.

When the modified hCNTF comprises at least one modification within the peptide of SEQ ID NO:14 (amino acids 87-101), or the equivalent peptide of an hCNTF variant, at least one modification is at Ala88, Leu90 and/or Gln95. While these amino acids may be substituted with any amino acid, in one preferred embodiment, Ala88→Asn, Leu→90Ala, and Gln95→Ala.

When the modified hCNTF comprises at least one modification within the peptide of SEQ ID NO:16 (amino acids 109-123), or the equivalent peptide of an hCNTF variant, at least one modification is at Leu112, preferably Leu112Ala.

When a modified hCNTF comprises at least one modification within the peptide of SEQ ID NO:24 (amino acids 163-175), or the equivalent peptide of an hCNTF variant, at least one modification is at Leu165, Trp168, Arg171, and His164. In one preferred embodiment, Leu165 is replaced with any one of Ala, Lys, Asn, Gln, Ser, Thr, Glu, Pro, Arg, Asp, Gly, His, or Cys. Most preferably, the Leu165→Ala. In another preferred embodiment, the His164→Lys, Glu, Ala, Ile, Leu, Trp, Tyr, Asp, GIn, Ser, or Thr. In a most preferred embodiment, His164→Lys, Glu, Ala, or Asp. In yet another preferred embodiment, Arg171→Ala, Lys, or Glu.

In a second embodiment, a modified hCNTF molecule having reduced antigenicity comprises a molecule modified by one or more of the modifications of group II comprising replacing the B or C helix region of human CNTF with the analogous region from a four helical bundle member, such as for example, interleukin-6 (IL-6), granulocyte colony stimulating factor (GCSF), IL-11, erythropoietin (EPO), leukemia inhibitory factor (LIF). The B helix of hCNTF is generally accepted to be found at approximately about amino acids 69-95 and the C helix is generally accepted to be found at approximately about amino acids 105-130.

In one specific embodiment of the modification group II of the invention, all or a portion of the B helix of hCNTF are replaced with all or a portion of the comparable region from IL-6, for example, all or a portion of Glu Glu Thr Cys Leu Val Lys Ile Ile Thr Gly Leu Leu Glu Phe Glu Val Tyr Leu Glu Tyr Leu (SEQ ID NO:25) of IL-6 and/or all or a portion of the C helix of hCNTF are replaced with all or a portion of the comparable region from IL-6, for example, all or a portion of Arg Ala Val Gln Met Ser Thr Lys Val Leu Ile Gln Phe Leu (SEQ ID NO:26). Similarly, improved molecules can be made as chimeric molecules substituting CNTF helix B amino acids with GSCF amino acids Ala Gly Cys Leu Ser Gln Leu His Ser Gly Leu Phe Leu Tyr Gln Gly Leu Leu Gln Ala (SEQ ID NO:27) or helix C sequences Pro Thr Leu Asp Thr Leu Gln Leu Asp Val Ala Asp Phe Ala Thr Thr Ile Trp Gln Gln Met Glu Glu Leu (SEQ ID NO:28).

A third embodiment of the invention are hCNTF, a variant of hCTNF, or a modified hCNTF molecule of the invention comprising a modification selected from group III with one or more added glycosylation site(s), which can modify proteolytic cleavage and antigen presentation of fragments. The modification may be one or more of the modifications selected from the group consisting of Ala1Asn, Ser18Asn, Asp30Asn, Ala33Asn, Asn47Ser, Asn49Ser, Ala59Asn, Glu66Asn, Glu92Asn, His97Asn, Thr99Asn, Ala139Ser, Glu164Asn, Gln167Asn, Val170Asn, Phe178Asn, His182Asn. In specific embodiment, a combinations of modifications, such as Gln74Asn+Asn76Ser, His84Asn+Leu86Ser, Leu86+Ala88Ser, etc., are included in a molecule exhibiting decreased antigenicity.

Pegylation of proteins has been shown to increase in vivo potency by enhancing stability and bioavailability while minimizing immunogenicity. It is known that the properties of certain proteins can be modulated by attachment of polyethylene glycol (PEG) polymers, which increases the hydrodynamic volume of the protein and thereby slows its clearance by kidney filtration. (See, e.g. Clark et al. (1996) J. Biol. Chem. 271: 21969-21977). U.S. Pat. No. 6,680,291 Wiegand et al., herein specifically incorporated by reference in its entirety, describes pegylated CNTF and CNTF variants. Accordingly, in specific embodiments, the hCTNF molecule and variants of the invention may be pegylated.

In a third embodiment of the invention, hCNTF, a variant of hCTNF, or a modified hCNTF molecule of the invention a modification from group IV comprising a fusion component (F) selected from the group consisting of a multimerizing component, a serum protein, or a molecule capable of binding a serum protein. In specific embodiments, the hCNTF, hCNTF variant, or modified hCTNF molecule of the invention may include multiple F components. When F is a multimerizing component, it includes any natural or synthetic sequence capable of interacting with another multimerizing component to form a higher order structure, e.g., a dimer, a trimer, etc. The multimerizing component may be selected from the group consisting of one or more of (i) a multimerizing component, optionally comprising a cleavable region (C-region), (ii) a truncated multimerizing component, (iii) an amino acid sequence between 1 to about 500 amino acids in length, optionally comprising at least one cysteine residue, (iv) a leucine zipper, (v) a helix loop motif, and (vi) a coil-coil motif.

In specific embodiments, the multimerizing component comprises one or more of an immunoglobulin-derived domain from, for example, human IgG, IgM or IgA. In specific embodiments, the immunoglobulin-derived domain is selected from the group consisting of the Fc domain of IgG, the heavy chain of lgG, and the light chain of IgG. The Fc domain of IgG may be selected from the isotypes IgG1, IgG2, IgG3, and IgG4, as well as any allotype within each isotype group. The invention further encompasses derivatives of an IgG component, for example, modified for specifically desired properties. In a preferred embodiment, the hCNTF, hCNTF variant, or modified hCTNF molecule of the invention includes one or two Fc domain(s) of human IgG1. In one specific embodiment of a group III modified molecule of the invention, the hCNTF variant Axokine™ (SEQ ID NO:3-4) is fused to a human Fc domain and exhibits improved properties of PK and stability.

The isolated nucleic acid molecule of the invention may further optionally comprise a signal sequence (SS) component. When a SS is part of the polypeptide, any SS known to the art may be used, including synthetic or natural sequences from any source, for example, from a secreted or membrane bound protein.

The modified hCNTF molecule having reduced antigenicity relative to a non-modified hCNTF molecule encompasses any molecule comprising one or more of the above-identified modifications from groups I, II, III or IV. Thus, the hCNTF molecule may be a naturally occurring hCNTF molecule (SEQ ID NO:1), or a modified hCNTF molecule. In more specific embodiments, the CNTF molecule is a modified hCNTF comprising a modification at one or more positions including: a substitute amino acid at position 17, a substitute amino acid at position 63, and a deletion of 13-20 amino acids at the carboxy terminus. More specifically, the modified hCNTF molecule a substitution at positions 17 and 63, and a deletion of 13-16 amino acids at the carboxy terminus. Even more specifically, the modified CNTF molecule is Axokine™, comprising the amino acid sequence of SEQ ID NO:3-4 (Ax-15) or SEQ ID NO:5 (Ax-13).

In a related second aspect, the invention features a nucleic acid molecule encoding a modified human CNTF (hCNTF) molecule of the invention, wherein the modified hCNTF is characterized by reduced antigenicity and/or improved properties such as stability or PK relative to a non-modified hCNTF molecule. In preferred embodiments, the nucleic acid molecule of the invention encodes hCNTF modified as described above.

In a third aspect, the invention encompasses vectors comprising the nucleic acid molecules of the invention, including expression vectors comprising the nucleic acid molecules operatively linked to an expression control sequence.

In a fourth aspect, the invention features host-vector systems for the production of a fusion polypeptide which comprise the expression vector, in a suitable host cell; host-vector systems wherein the suitable host cell is, without limitation, a bacterial, yeast, insect, or mammalian cell. Examples of suitable cells include E. coli, B. subtilis, BHK, COS and CHO cells. Additional encompassed are modified hCNTF molecules of the invention modified by acetylation or pegylation. Methods for acetylating or pegylating a protein are well known in the art.

In a fifth related aspect, the invention features a method of producing a modified hCNTF molecule of the invention, comprising culturing a host cell transfected with a vector comprising a nucleic acid sequence of the invention, under conditions suitable for expression of the protein from the host cell, and recovering the polypeptide so produced.

The reduced antigenicity or improved modified hCNTF molecules of the invention are therapeutically useful for treating any disease or condition which is improved, ameliorated, or inhibited by treatment with CNTF. In a sixth aspect, the invention feature a pharmaceutical composition, comprising a modified hCNTF molecule having a reduced antigenicity relative to a non-modified hCNTF molecule, and a pharmaceutically acceptable carrier.

In a seventh aspect, the invention features a method of treating obesity, and diseases related to obesity, comprising administering to a human subject a pharmaceutical composition of the invention, comprising a modified hCNTF molecule having reduced antigenicity and/or improved properties such as stability or PK. The method of the invention includes treatment of diseases related to obesity, such as non-insulin dependent diabetes mellitus.

In one specific embodiment, the method of treating diseases related to obesity comprises treating non-insulin dependent diabetes mellitus (NIDDM) comprising administering to a subject in need thereof the pharmaceutical composition of the invention. In specific embodiments, a modified hCNTF having reduced antigenicity or other improved properties is administered in combination with one or more therapeutic agents, for example, a second modified CNTF molecule, a thiazolidinedione, CB1 antagonists, Meridia, Orlistat, etc.

In an eighth aspect, the invention features a method of preventing or decreasing weight gain. In a seventh aspect, the invention features a method of treating obesity, and diseases related to obesity, comprising administering to a human subject a pharmaceutical composition of the invention, comprising a modified hCNTF molecule having reduced antigenicity or other improved properties.

Other objects and advantages will become apparent from a review of the ensuing detailed description.

### DETAILED DESCRIPTION OF THE INVENTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only the appended claims.

As used in this specification and the appended claims, the singular forms “a”, “an”, and “the” include plural references unless the context clearly dictates otherwise. Thus for example, a reference to “a method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference in their entirety.

Definitions

By the term “reduced antigenicity” is meant a molecule having a reduction in, for example, the T cell assay described herein that reflects the reduction of antibody response in patients treated with the molecule. The removal of antibody epitopes from of a modified hCNTF of the invention may be determined in a variety of ways known to the art, including in a direct binding assay and competition experiment between a modified hCNTF molecule of the invention and native hCNTF, using human or animal sera collected from a subject that produced antibodies after treatment with a modified hCNTF molecule of the invention. See, for example, U.S. Pat. No. 6,309,873, herein specifically incorporated by reference in its entirety, which describes an assay for determining antibody epitope removal. For an example of a method for determining the T cell-Dendritic cells response and potential antigenicity of a molecule, see Example 1 below.

The term “hCNTF molecule”, “hCNTF variant”, or “modified hCNTF molecule” encompass molecules with contain at least one modification defined in modification groups I, II, III or IV above, and which (1) retain CNTF activity as measured by in vitro assay (described below) or in vivo assay as described in, for example, U.S. Pat. No. 6,309,873, and (2) exhibit reduced immunogenity (measured as described below) and/or one or more improved properties, such as improved in vitro or in vivo stability.

Nucleic Acid Constructs and Expression

The present invention provides for the construction of nucleic acid molecules encoding modified human CNTF molecules having reduced antigenicity. These nucleic acid molecules are inserted into a vector that is able to express the modified human CNTF molecules having reduced antigenicity of the invention when introduced into an appropriate host cell. Appropriate host cells include, but are not limited to, bacterial, yeast, insect, and mammalian cells. Any of the methods known to one skilled in the art for the insertion of DNA fragments into a vector may be used to construct expression vectors encoding the modified human CNTF molecules of the invention under control of transcriptional and/or translational control signals.

Expression of the nucleic acid molecules of the invention may be regulated by a second nucleic acid sequence so that the molecule is expressed in a host transformed with the recombinant DNA molecule. For example, expression may be controlled by any promoter/enhancer element known in the art. Promoters which may be used to control expression of the chimeric polypeptide molecules include, but are not limited to, a long terminal repeat (Squinto et al. (1991) Cell 65:1-20); SV40 early promoter region, CMV, M-MuLV, thymidine kinase promoter, the regulatory sequences of the metallothionine gene; prokaryotic expression vectors such as the beta-lactamase promoter, or the tac promoter (see also Scientific American (1980) 242:74-94); promoter elements from yeast or other fungi such as Gal 4 promoter, ADH, PGK, alkaline phosphatase, and tissue-specific transcriptional control regions derived from genes such as elastase I.

Expression vectors capable of being replicated in a bacterial or eukaryotic host comprising the nucleic acid molecules of the invention are used to transfect the host and thereby direct expression of such nucleic acids to produce the modified human CNTF molecules of the invention. Transfected cells may transiently or, preferably, constitutively and permanently express the polypeptides of the invention

The modified human CNTF molecules of the invention may be purified by any technique known in the art. See, for example, U.S. Pat. No. 5,349,056, herein specifically incorporated by reference in its entirety. For example, and not by way of limitation, the factors may be recovered from cells either as soluble proteins or as inclusion bodies, from which they may be extracted quantitatively by 8M guanidinium hydrochloride and dialysis (see, for example, U.S. Pat. No. 5,663,304). In order to further purify the factors, conventional ion exchange chromatography, hydrophobic interaction chromatography, reverse phase chromatography or gel filtration may be used.

Determination of Affinity to CNTFRa

The modified human CNTF molecules of the invention specifically bind CNTF receptor a (CNTFRa) with an affinity of at least equal to that of native CNTF, as measured in a TF1-CNTFRa growth assay. Briefly, TF1-CNTFRa cells were created by transfecting TF1 cells with a retrovirus expressing CNTFRa. Stable cell lines were isolated that grow in response to CNTR stimulation. Alternatively, affinity can be measured by a solid phase binding assay (see, for example, U.S. Pat. No. 6,565,869 Ciliberto et al., herein specifically incorporated by reference in its entirety).

Fusion Components

In specific embodiments, the hCNTF molecules of the invention comprise one or more fusion (F) component(s) which may be the same or different. In specific embodiments, the fusion component may be selected from the group consisting of a multimerizing component, a serum protein, or a molecule capable of binding a serum protein. When F is a multimerizing component, it includes any natural or synthetic sequence capable of interacting with another multimerizing component to form a higher order structure, e.g., a dimer, a trimer, etc. The multimerizing component may be selected from the group consisting of (i) a multimerizing component, optionally comprising a cleavable region (C-region), (ii) a truncated multimerizing component, (iii) an amino acid sequence between 1 to about 500 amino acids in length, (iv) a leucine zipper, (v) a helix loop motif, and (vi) a coil-coil motif. When F is a multimerizing component comprising an amino acid sequence between 1 to about 500 amino acids in length, the sequence may contain one or more cysteine residues capable of forming a disulfide bond with a corresponding cysteine residue on another fusion polypeptide comprising an F with one or more cysteine residues.

In a preferred embodiment, the multimerizing component comprises one or more immunoglobulin -derived domain from human IgG, IgM or IgA. In specific embodiments, the immunoglobulin-derived domain is selected from the group consisting of the Fc domain of IgG, the heavy chain of IgG, and the light chain of IgG. The Fc domain of IgG may be selected from the isotypes IgG1, IgG2, IgG3, and IgG4, as well as any allotype within each isotype group. In a preferred embodiment, F is the Fc domain of IgG1, or a derivative thereof which may be modified for specifically desired properties. In specific embodiments, the hCNTF molecules of the invention comprises one or two Fc domain(s) of IgG1.

In one embodiment, the F is a serum protein or fragment thereof, is selected from the group consisting of α-1-microglobulin, AGP-1, orosomuciod, α-1-acid glycoprotein, vitamin D binding protein (DBP), hemopexin, human serum albumin (hSA), transferrin, ferritin, afamin, haptoglobin, α-fetoprotein thyroglobulin, α-2-HS-glycoprotein, β-2-glycoprotein, hyaluronan-binding protein, syntaxin, C1R, C1q a chain, galectin3-Mac2 binding protein, fibrinogen, polymeric Ig receptor (PIGR), α-2-macroglobulin, urea transport protein, haptoglobin, IGFBPs, macrophage scavenger receptors, fibronectin, giantin, Fc, α-1-antichyromotrypsin, α-1-antitrypsin, antithrombin III, apolipoprotein A-I, apolipoprotein B, β-2-microglobulin, ceruloplasmin, complement component C3 or C4, Cl esterase inhibitor, C-reactive protein, cystatin C, and protein C. In a more specific embodiment, F is selected from the group consisting of α-1-microglobulin, AGP-1, orosomuciod, α-1-acid glycoprotein, vitamin D binding protein (DBP), hemopexin, human serum albumin (hSA), afamin, and haptoglobin. The inclusion of an F component may extend the serum half-life of the hCNTF molecule of the invention when desired. See, for example, U.S. Pat. Nos. 6,423,512, 5,876,969, 6,593,295, and 6,548,653, herein specifically incorporated by reference in their entirety, for examples of serum albumin fusion proteins. hSA is widely distributed throughout the body, particularly in the intestinal and blood components, and has an important role in the maintenance of osmolarity and plasma volume. It is slowly cleared in the liver, and typically has an in vivo half-life of 14-20 days in humans (Waldmann et al. (1977) Albumin, Structure Function and Uses; Pergamon Press; pp. 255-275).

When F is a molecule capable of binding a serum protein, the molecule may be a synthetic small molecule, a lipid or liposome, a nucleic acid, including a synthetic nucleic acid such as an aptomer, a peptide, or an oligosaccharide. The molecule may further be a protein, such as, for example, FcγR1, FcγR2, FcγR3, polymeric Ig receptor (PIGR), ScFv, and other antibody fragments specific for a serum protein.

Pegylated Modifications

In one embodiment of the invention, a modified hCNTF molecule may further be pegylated by attachment of polyethelyne glycol (PEG) polymers, to increase stability and bioavailability while minimizing immunogenicity. Pegylation may be accomplished by one versed in the art by several methods. In particular, modification of a polypeptide amino terminus or side chain or side chains of one or more different amino acid residue types including but not limited to lysines, histidines, arginines, tyrosines, glutamic acids and aspartic acids can be accomplished by adding one or more PEG moieties randomly to the protein. Specificity and control of the reaction can be accomplished although not always to homogeneity. Alternatively, since CNTF and certain CNTF variants lack any cysteine within the primary amino acid sequence, one or more cysteines can be engineered into specific sites within the amino acid sequence of the protein, thereby providing a specific site of attachment for one or more PEG moiety. Addition of PEG in a controlled manner can provide decreased immunogenicity, increased pharmacokinetic half-life, decreased proteolytic cleavage susceptibility, increased protein stability, and decreased T-cell recognition of modified peptides. An example includes but is not limited to Glu92Cys within the B-helix of a CNTF variant which provides for a biologically active protein. Specific sites for cysteine incorporation exist throughout the CNTF structure and include but are not limited to alpha helical domain amino acids which are externally facing based on crystal structure analysis as well as major loop structures such as the AB loop and the CD loop.

Therapeutic Uses

The modified human CNTF molecules of the invention are therapeutically useful for treating any disease or condition which is improved, ameliorated, inhibited or prevented by treatment with CNTF. More specifically, the modified human CNTF molecules of the invention are therapeutically useful for the treatment of obesity or obesity-related conditions, in a human subject suffering therefrom, including non-insulin dependent diabetes mellitus (NIDDM), as well as hyperlipidemia, hyperinsulinemia, hyperglycemia associated with metabolic syndrome and NIDDM. The modified human CNTF molecules of the invention are further therapeutically useful in the treatment of hepatic steatosis, decreased gallbladder motility, gall stone formation, and sleep apnea.

Combination Therapies

In numerous embodiments, the modified human CNTF molecules of the invention may be administered in combination with one or more additional compounds or therapies. For example, multiple modified human CNTF molecules can be co-administered[TJD1], or one or molecules can be administered in conjunction with one or more therapeutic compounds. A benefit of the combined use of the modified human CNTF molecules the invention with a second therapeutic agent is that may provide improved efficacy and/or reduced toxicity of either therapeutic agent.

Preferred therapeutics for combining with the modified CNTF molecules of the invention include therapeutics used to treat obesity, obesity-related conditions, and type II diabetes, such as sulfonylurea, biguanide metformin (e.g., Glucophage™, Bristol-Myers Squibb), and metformin variants, alpha-glucosidase inhibitors (e.g., Glucobay™, Precose™, Bayer), a thiazolidinedione such as troglitazone (Rezulin™, Warner-Lambert), rosiglitazone (Avandia™, SmithKline Beecham), pioglitazone (Actos™, Takeda/Lilly), repaglintide (NovoNorm™, Prandin™, Novo Nordisk), a small molecule such as MCC-555 (Mitsubishi), Targretin™ (Ligand Pharmaceuticals), bromocriptine (Ergoset™, Ergo Science), 5HT_{2c} receptor agonists (Cerebrus™, Roche), sibutramine, (Meridia™, Knoll), orlistat (Xenical™, Roche), leptin pathway therapeutics (metreleptin, Amgen), ghrelin antagonists, neuropeptide receptor antagonists, thermogenesis pathway therapeutics, PPARγ antagonists, aminoguanidine, AGE inhibitors, pimagedine, symlin (Pramlintide™, Exendin-4™, GLP-1, Amylin), HNF-4 modulators (Lingand Pharmaceuticals), MC4-R receptor modulators and other GPCRs (Millenium), small molecule MC4-R agonists, UCP modulators, and rimonabant (Acomplia™, Sanofi-Aventis) and other endocannabinoid receptor antagonists, bupropion (Wellbutrin™, Zyban™, Sanofi-Aventis), miglitol (Glyset™, Bayer), zonisamide (Zonegran™, Dainippon) and other calcium channel antagonists, topirqamate (Topamax™, Johnson & Johnson), bombesin, CCK-A agonists (GSK), and beta-3 AR agonists (L796568, Merck).

Methods of Administration

The invention provides methods of treatment comprising administering to a subject an effective amount of a modified human CNTF molecule of the invention. In a preferred aspect, the modified human CNTF molecule is substantially purified (e.g., substantially free from substances that limit its effect or produce undesired side-effects). The subject is preferably a mammal, and most preferably a human.

Various delivery systems are known and can be used to administer an agent of the invention, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432), construction of a nucleic acid as part of a retroviral or other vector, etc. Methods of introduction can be enteral or parenteral and include but are not limited to intradermal, intramuscular, intraperitoneal, intravenous, subcutaneous, intranasal, intraocular, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. Administration can be acute or chronic (e.g. daily, weekly, monthly, etc.) or in combination with other agents. Pulmonary administration can also be employed, e.g., by use of an inhaler or nebulizer, and formulation with an aerosolizing agent.

In another embodiment, the active agent can be delivered in a vesicle, in particular a liposome, in a controlled release system, or in a pump. In another embodiment where the active agent of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see, for example, U.S. Pat. No. 4,980,286), by direct injection, or by use of microparticle bombardment, or coating with lipids or cell-surface receptors or transfecting agents, or by administering it in linkage to a homeobox-like peptide which is known to enter the nucleus (see e.g., Joliot et al. (1991) Proc. Natl. Acad. Sci. USA 88:1864-1868), etc. Alternatively, a nucleic acid can be introduced intracellularly and incorporated within host cell DNA for expression, by homologous recombination.

In a specific embodiment, it may be desirable to administer the pharmaceutical compositions of the invention locally to the area in need of treatment; this may be achieved, for example, and not by way of limitation, by local infusion during surgery, topical application, e.g., by injection, by means of a catheter, or by means of an implant, the implant being of a porous, non-porous, or gelatinous material, including membranes, such as sialastic membranes, fibers, or commercial skin substitutes.

A composition useful in practicing the methods of the invention may be a liquid comprising an agent of the invention in solution, in suspension, or both. The term “solution/suspension” refers to a liquid composition where a first portion of the active agent is present in solution and a second portion of the active agent is present in particulate form, in suspension in a liquid matrix. A liquid composition also includes a gel. The liquid composition may be aqueous or in the form of an ointment.

Pharmaceutical Compositions

The present invention also provides pharmaceutical compositions comprising a human CNTF molecule of the invention. Such compositions comprise a therapeutically effective amount of one or more human CNTF molecules, and a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical carriers are described in “Remington's Pharmaceutical Sciences” by E. W. Martin.

The human CNTF molecules of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the human CNTF molecule that will be effective for its intended therapeutic use can be determined by standard clinical techniques based on the present description. In addition, in vitro assays may optionally be employed to help identify optimal dosage ranges. Generally, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems. The amount of compound administered will, of course, be dependent on the subject being treated, on the subject's weight, the severity of the affliction, the manner of administration, and the judgment of the prescribing physician. The therapy may be repeated intermittently while symptoms are detectable or even when they are not detectable.

Cellular Transfection and Gene Therapy

The present invention encompasses the use of nucleic acids encoding the human CNTF molecules of the invention for transfection of cells in vitro and in vivo. These nucleic acids can be inserted into any of a number of well-known vectors for transfection of target cells and organisms. The nucleic acids are transfected into cells ex vivo and in vivo, through the interaction of the vector and the target cell. The compositions are administered (e.g., by injection into a muscle) to a subject in an amount sufficient to elicit a therapeutic response. An amount adequate to accomplish this is defined as “a therapeutically effective dose or amount.”

For gene therapy procedures in the treatment or prevention of human disease, see for example, Van Brunt (1998) Biotechnology 6:1149-1154.

### SPECIFIC EMBODIMENTS

Studies described below show that immunogenicity can result from protein structure, as well as from purification artifacts such as aggregate formation. Accordingly, reduction of immunogenicity is achieved by specific changes in the amino acid structure of hCNTF, including specific amino acid substitutions, substitutions of multiple amino acids with analogous regions from related proteins known to be non-immunogenic, and by reducing aggregate formation. Further, desirable properties can be conferred on hCNTF by the addition of a fusion component such as fusion to an Fc domain. Increased pharmacokinetic half-life may result in overall decreased dosing levels which would result in decreased availability to T-cell surveillence and therefore decreased immunogenicity.

### EXAMPLES

The following example is put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### T-Cell Assay for Antigenicity

There are several modifications of the ex vivo assay used to detect antigenicity of a protein. Generally, human blood samples are drawn and peripheral blood cells are isolated. Blood donors can either be from Test Protein (a human CNTF molecule of the invention, including modified hCNTF with reduced antigenicity or Axokine™-Fc fusion proteins) treated patients or from a panel of donors with a diversity of MHCII haplotypes. One example of an antigenicity assay, is to culture dendritic and CD4⁺ T cells isolated from the human blood under standard cell culture conditions in the presence of the Test Protein. This expands the T populations that are specific for the Test Protein, if they are present. These dendritic and T cells are then mixed together into individual wells of a tissue culture plate along with 9-13 amino acid peptides whose sequences redundantly cover the full sequence of the Test Protein. Each well of a plate receives a different peptide. Control wells receive full length Test Protein. After culturing for 1-2 days, the T cells can either be assayed for proliferation using ³H-thymidine or by ELISPOT analysis for interferon gamma and/or IL-4 secretion. Those mixtures that produce a signal over background indicate the added peptide are antigenic. Peptides modified as described above are tested for reduced antigenity, and full length modified CNTF proteins are generated which include the modifications resulting in reduced antigenicity. Alternatively, a panel of mice that are transgenically modified to express the various common human MHC class II haplotypes can be generated and use to test both the antibody responses, as well as the T cell response, to administration of the Test Protein.

### Example 2

### Cell-Based Assay to Assess CNTF Activity

A variety of methods for determining if a modified CNTF molecule retains CNTF activity are known to the art. For example, U.S. Pat. Nos. 5,349,056, 6,472,178, and Mosmann (1983) J. Immunol. Methods 65:55-63, both of which publications are herein specifically incorporated by reference in their entirety, describe methods for measuring the biological activity of modified CNTF molecules.

### Example 3

### In Vivo Assay to Determine Antigenicity

A TF1-CNTFRa growth assay was developed by transfection of TF1 cells with a retrovirus that expresses both the CNTFR-a receptor and eGFP. Transfected cells were selected by FACs analysis for eGFP, and a single cell line was identified that proliferated when CNTF was added. The assay is performed by plating TF1-CNTFR cells at 25,000 cells per well. Serial dilutions starting at 5 ng/ml of CNTF, or modified versions, are added to the wells containing the cells. After 3 days of incubation at 37° C. in a CO2 incubator, the tetrazolium salt MTS is added and OD490 is measured several hours afterward. Activity of the protein is determined as the concentration of protein that stimulated half of the maximal response (EC50).

### Example 4

### Immunogenicity Resulting from Protein Aggregation

Studies conducted with purified Axokine™ protein identified amino acids susceptible to cleave during purification and/or storage causing protein aggregation linked to increased immunogenity in patients and reduced efficacy. High molecular weight aggregates were found to be generated from shear force, e.g., during filtration, which contain fragments of degraded protein. Multiple filtrations were found to lead to the accumulation of degraded species in the high-molecular weight fractions. Aggregates were analyzed by SDS-PAGE electrophoresis (FIG. 1). The results showed that band A (4097 Da) contained Axokine™ fragments cleaved at positions 2, 6, and 13 of the Axokine™ protein; band B (5315 Da) contained fragments cleaved at amino acids 2 and 6; band C (11852 Da) contained fragments cleaved at amino acids 2 and 83; band D (13425 Da) contained fragments cleaved at amino acids 70 and 74; band E (15086 Da) contained fragments cleaved at positions 55 and 59; band F (15815 Da) contained fragments cleaved at amino acids 48 and 52; band G (21112 Da) contained fragments cleaved at amino acids 2 and 6; bands J, I, and H did not contain Axokine™ fragments. Additionally, further studies found aggregate resulting from dimer formation resulting from interaction between specific amino acid candidates, for example Tyr at position 132 of SEQ ID NO:3 (position 131 of SEQ ID NO:4).

### Example 5

### Immunogenicity of Axokine™ Variants

Seven week old male and female ICR mice (Taconic) were treated (0.05 mg/kg, subcutaneous injection, 5 ul/g twice per week) with Axokine™ (Ax) (control) (group A), Ax-Aggregate Free (group B), Ax-Norleucine Variant Free (group C), and Ax-Degraded (stored 2 weeks at 37° C.) (group D). Blood samples were collected at day 0 (before treatment), 14, 32, and 63 (1 hr after injection) and analyzed for the presence of anti-Akokine™ antibodies. Spleens from the control group were analyses for MHC. The Ax-Aggregate Free material (group B) was Axokine™ subjected to rigorous purification such that virtually no aggregate formation was present. Norleucine is a potential immunogenic amino acid variant which has been shown to be inserted in the pace of methionine in E. coli. The protein of group C was purified to remove all norleucine, however it possessed a very high percentage of aggregated protein.

The results showed that 44% (8/20) of control animals were negative for antibodies at day 14, and remained negative at day 32 (7/20), but only 20% remained negative by day 63 (4/20). Animals positive at day 14 exhibited an overall weight gain throughout the rest of the experimental period, whereas animals that become antibody positive at later time points, maintained weight loss. For animals receiving the Ax-Aggregate Free material (group B), animals negative at day 14 (55%; 11/20) generally remained negative and continued to lose weight through out the experimental period (45%; 9/20). For group C animals treated with Norleucine Variant Free Axokine™ (high aggregate), 45% (9/20) animals tested negative for antibodies at day 14, 1/20 at day 32, and all were positive by day 63. After an initial weight loss, most of these animals appeared to gain weight throughout the experimental period regardless of when they developed antibodies. 35% (7/20) animals in group D were negative for antibodies at day 14, which declined to 25% (5/20) at day 32, and 10% (2/20) at day 63.

1. A modified human CNTF (hCNTF) molecule or variant thereof having reduced antigenicity relative to a non-modified hCNTF molecule or variant thereof.

2. The modified hCNTF molecule of claim 1, wherein the hCTNF molecule or variant molecule modified is selected from the group consisting of SEQ ID NOs:1-7.

3. The modified hCTNF molecule of claim 2, wherein the hCTNF variant molecule modified is Axokine™ (SEQ ID NO:3 or 4).

4. The modified hCNTF molecule of claim 3, wherein the modification is one or more changes peptides of SEQ ID NOs:9-24.

5. The modified hCNTF molecule of claim 4, wherein the modification is s substitution at one or more of Pheb 105, His110, Leu112 and/or Leu113.

6. The modified hCNTF molecule of claim 4, wherein the at least one modification is within the peptide of SEQ ID NO:13 (amino acids 80-94).

7. The modified hCNTF molecule of claim 6, wherein the at least one modification is at Tyr80, Phe 83, Ala88 and/or Leu90.

8. The modified hCNTF molecule of claim 4, wherein the at least one modification is within the peptide of SEQ ID NO:14 (amino acids 87-101).

9. The modified hCNTF molecule of claim 8, wherein the at least one modification is at Ala88, Leu90 and/or Gln95.

10. The modified hCNTF molecule of claim 4, wherein the least one modification is within the peptide of SEQ ID NO:16 (amino acids 109-123).

11. The modified hCNTF molecule of claim 10, wherein the at least one modification is at Leu112.

12. The modified hCNTF molecule of claim 4, wherein the least one modification is within the peptide of SEQ ID NO:24 (amino acids 163-175).

13. The modified hCNTF molecule of claim 12, wherein at least one modification is at Leu165, Trp168, Arg171, and/or His164.

14. The modified hCNTF molecule of claim 3, wherein the modification comprises replacing a helix region of hCNTF with an analogous region from a four helical bundle member.

15. The modified hCNTF of claim 14, wherein the four helical bundle member is selected from the group consisting of interleukin-6 (IL-6), granulocyte colony stimulating factor (GCSF), IL-11, erythropoietin (EPO), and leukemia inhibitory factor (LIF).

16. The modified hCNTF molecule of claim 4, wherein the modification comprises replacing a helix region of hCNTF with an analogous region from a four helical bundle member.

17. The modified hCNTF molecule of claim 3, wherein the modification comprises adding one or more glycosylation site(s).

18. The modified hCNTF molecule of claim 3, wherein the modified molecule further comprises a fusion component selected from the group consisting of a multimerizing component, a serum protein, or a molecule capable of binding a serum protein.

19. The modified hCNTF molecule of claim 18, wherein the multimerizing component comprises one or more immunoglobulin-derived domain(s).

20. The modified hCNTF molecule of claim 19, wherein the immunoglobulin-derived domain is an Fc domain of IgG is selected from the isotypes IgG1, IgG2, lgG3, and IgG4.
